(19) United States
(12) Patent Application Publication
(10) Pub. No.: US 2024/0285843 A1
(43) Pub. Date: Aug. 29, 2024

(54) FLUID DRAINAGE MONITORING, MANAGEMENT, AND CONTROL SYSTEM, METHOD, AND DEVICE

(71) Applicant: TRANCE MEDICAL, LLC, Youngstown, OH (US)

(72) Inventor: FARZAD AHMADI, Youngstown, OH (US)

(21) Appl. No.: 18/444,707

(22) Filed: Feb. 18, 2024

Related U.S. Application Data
(60) Provisional application No. 63/447,932, filed on Feb. 24, 2023.

(51) Int. Cl. A61M 1/00; G16H 50/20; G16H 50/70

(52) U.S. Cl. CPC A61M 1/70; G16H 50/20; G16H 50/70; A61M 2205/123; A61M 2205/3303; A61M 2205/3327; A61M 2205/3334; A61M 2205/3368; A61M 2205/3379; A61M 2205/3553; A61M 2205/502; A61M 2230/50

(57) ABSTRACT

A real-time fluid drainage monitoring and control system, method, and device are disclosed, wherein the system is portable, standalone, and can be networked to local and global servers, and is amenable to operation for different medical needs including, but not limited to, external ventricular drain (EVD), external lumbar drain (ELD), and urine output (UO) monitoring and management.

This application claims priority to U.S. Ser. No. 63/447,932, filed on Feb. 24, 2023

### FIELD OF THE INVENTION

Fluid drainage in living human and non-human patients; more specifically, the invention is directed to the monitoring, management, and control thereof.

### BACKGROUND OF INVENTION

Fluid drainage has applications that are known and continue to have clinical relevance in the healthcare industry in many fields, including at least urology, neurology, cardiology, and radiology.

Cerebrospinal Fluid (CSF) drainage, one of the most common procedures performed in neurocritical settings, is bifurcated into CSF shunts and drainage systems, that is External Ventricular Drainage, EVD, (a temporary method for draining excess cerebrospinal fluid (CSF) from the ventricles in the brain or the spinal cord, both of which are surrounded by CSF), and External Lumbar Drainage, ELD, (a closed sterile temporary system, which allows drainage of cerebrospinal fluid (CSF) from the lumbar subarachnoid space).

The EVD hardware is typically emplaced by making a frontal incision along the scalp and drilling a burr hole through the skull, through which a catheter is passed into the ipsilateral brain ventricle. EVDs typically serve three functions: monitoring Intracranial Pressure (ICP), draining CSF to treat hydrocephalus (i.e., fluid buildup in brain ventricles), and rapidly reducing ICP. Indications for placement of an EVD include but are not limited to hydrocephalus following brain tumor surgery, drainage of infected CSF (e.g., meningitis, ventriculitis), severe traumatic brain injury, and reduced Glasgow Coma Score.

The common use of ELD within the Neurosciences Unit includes for treatment of communicating hydrocephalus (e.g., in subarachnoid hemorrhage). ELD placements, drainage via a tube that is placed in the lumbar spine, can function as a temporizing measure before definitive neurosurgical intervention. Indications for ELD placement include, but are not limited to, CSF leakage in the brain, posterior fossa lesions, shunt infections, thoracic endovascular aortic repair procedures, and sometimes, idiopathic intracranial hypertension.

Many of these indications for EVD/ELD placement are associated with raised ICP (>20 mmHg) secondary to CSF outflow obstruction resulting in hydrocephalus. In standard systems known in the art, once the EVD/ELD is inserted, a desired drain height or pressure is determined for the collection system for a given patient. Infection, occlusion, over-drainage, and under-drainage are the most common risks for EVDs/ELDs placement. Among them, one of the most frequently occurring issues in clinical practice is the occlusion of EVD/ELD catheters, which can occur due to cellular debris, blood clots, or brain tissue fragments. Signs indicating catheter obstruction include ICP waveform dampening and a decline in CSF flow. If promptly noticed, manual action can be taken, but exigencies of healthcare treatment often divert what should be constant attention to these parameters to ensure they remain within physiologically acceptable ranges.

Acute Kidney Infection (AKI) is another clinical application in which monitoring, management and control of fluid drainage parameters within pre-set physiological parameters is critical. AKI causes a buildup of waste products in the blood as the kidney fails to maintain the correct balance of fluid in the body. This is reflected in altered Urinary Output (UO), a key physiological parameter to be maintained within preset limits. Continuous monitoring and control over patients' UO, often for days on end, is required to determine the cause of AKI. This imposes the burden of requiring near constant monitoring by healthcare staff, while placing patients at risk should a key parameter divert from acceptable without being either noticed in time, alarmed in time, or promptly controlled, to avoid adverse sequelae.

Abnormal fluid color, e.g., cloudy fluid, opaque color, and the like, are critical indicators of morbidity onset in clinical settings. A cloudy and reddish fluid noted during UO monitoring may indicate the presence of infection, blood, or both. Time-consuming and expensive laboratory tests often delay the diagnosis of AKI or CSF infection, often leaving patients at risk and in extremis in the interim.

Nurses are primarily allocated responsibility for monitoring, maintaining, and troubleshooting known fluid drainage systems. For example, it is standard practice for nurses to regularly record drainage parameters, e.g., CSF drainage parameters, UO parameters, and to manually enter such data into electronic health records (EHRs). Hourly EVD/ELD/UO monitoring significantly increases healthcare costs and nurses' workload, while at the same time, providing only intermittent monitoring, management, and control of drainage parameters. Continuous monitoring of patients ‘vital signs in Intensive Care Units (ICUs), and storage of data in EHRs, provide valuable clinical information to healthcare professionals. Accordingly, a major risk factor for patients undergoing fluid drainage in a clinical setting remains the need for a real-time, reliable, constant, monitoring, management, and control system, method, and device to intelligently manage such procedures.

The invention described in the present written disclosure addresses this long-felt need by providing an enabling teaching of how to make and use a fluid drainage management and control system, method, and device, which optimizes the care that can be provided in the healthcare fluid drainage setting, by providing a real-time, reliable, constant, fluid drainage, monitoring, and control system, method, and device, which intelligently manages such procedures.

### SUMMARY OF THE INVENTION

A real-time fluid drainage, monitoring, and control system, method, and device are disclosed. Preferably, the system is portable, standalone, and can be networked to local and global servers, and is amenable to operation for different medical needs including, but not limited to, external ventricular drain (EVD), external lumbar drain (ELD), and urine output (UO) monitoring and management. The system detects occlusion, over-drainage, under-drainage, and predicts the probability of infection and blood in fluid, and instantly alarms healthcare professionals. Data are stored and displayed on a monitoring console. Preferably, the system, method and device collects a large amount of data from many patients, learns from the database, and improves its performance. By harnessing the power of AI and learning algorithms on local and global servers, the system perpetually grows wiser with every monitoring. Such an AI-based system improves diagnosis, evaluates treatment efficacy, and unlocks clinically relevant information that assists clinical decision-making.

### BRIEF DESCRIPTION OF THE DRAWINGS

The drawings provided with this invention disclosure are for non-limiting, illustrative purposes only, to ensure a fully enabling and adequate written description of exemplary embodiments of the invention, without necessarily showing all possible implementations or embodiments. The scope of the invention should not be interpreted to be co-extensive with the drawings. Rather, for an apprehension of the scope of the invention, reference should be had to the appended claims.

FIG. 1 provides a depiction of an embodiment of the invention, 1 viewed from the front, which includes: 1) a monitoring console 103, including, preferably, a touch display, 112, selector, 107, for selecting a desired drainage procedure, such as UO, EVD, or ELD, power on/off switch 108, preferably located on the top of the panel for ease of access, monitoring, command, and control start, pause, and stop/reset selectors 109, 110, 111, respectively, in a preferred embodiment, are situated below touch display 112, wherein the device begins real-time measurements when initiated by activating the start button 109, which initiates operation based on the functionality chosen by selector 107; 2) at least one remote monitoring device, selected from the group consisting of, in this embodiment, a local server 114 and a global server 115 communicating via a wired or preferably wireless fidelity network (WiFi) 113 3) sterilized single-use drainage parts, including but not limited to catheters 100, 102, and 104, stopcock 101 and 122, pressure transducer 123, drainage cartridge 105, collection bag 106, which are user installable and replaceable.

FIG. 2 provides, in a side view of console 103, a more detailed schematic illustration of catheters 100, 102, and 104, pressure transducer 123, stopcock 122, electric motor 124, and collection bag 106 mounted to an IV pole 125 or similar support structure.

FIG. 3 provides, a side view of the monitoring console 103 with two mechanical mechanisms. Attached to the IV pole 125 or similar support structure, is a stationary circular gear 143, complete with bearings and a pin. The teeth of gear 143 interlocks with those of a moving rack 146. The rack 146 can only move linearly in a two-dimensional plane up or down and is confined to a specific track. The monitoring console 103 is attached to the rack 146. This mechanism just moves the monitoring console 103 up or down. There is a similar mechanical mechanism inside the base 145 to move the IV pole 125 or similar support structure up or down. In this case, a stationary circular gear 144, complete with bearings and a pin, rotates clockwise or counterclockwise as needed. The teeth of gear 144 interlock with those of a moving rack 147. The rack 147 can only move linearly up or down and is confined to a specific track. This mechanical mechanism moves the IV pole 125 or similar support structure and all attached components to it up or down.

FIG. 4 provides a schematic illustration of the rear view of monitoring console 103, with a rear enclosure removed to reveal processing unit 121, which, preferably, includes but is not necessarily limited to: a processor, on-board data storage, and a Wi-Fi module; electric motors 116, such as a servo motor, step motor, or the like, are in operative communication with stopcocks, valves and the like, such as 122 and 101, to monitor, manage, and control drainage fluid flow.

FIG. 5 provides a more detailed, schematic illustration, front view, visible to a user, of drainage cartridge 105.

FIG. 6 provides a top view of a preferably transparent drainage cartridge 105, heating element 119, catheter 102, and camera 118, the distance of which from drainage cartridge 105 is adjusted such that it is visible to the camera's field of view, such that, as fluid drains and accumulates in drainage cartridge 105 via catheter 102, drainage fluid flow rate and any discoloration of fluid is monitored, measured in real-time by the camera 118, Note that camera 118 and the heating element 119 are inside the enclosure of the monitoring console 103. The heating element 119 can be activated when the fluid inside the drainage cartridge 105 is not visible to camera 118. In this case, the camera 118 operates as a thermal camera and detects the higher fluid temperature than the air inside the drainage cartridge 105 to measure fluid flow rate and volume.

FIG. 7 is a schematic illustration of the back view of the monitoring console 103 as an alternative to the one in FIG. 2. In this approach, pinch valves 117 and 120 could be used instead of electric motors 116 and 124 to control the fluid flow.

FIG. 8 is a schematic illustration of the back view of the monitoring console 103. The pinch valves 117 and 120 are open and closed, respectively, to accumulate the fluid inside the drainage cartridge 105.

FIG. 9 is a schematic illustration of the back view of the monitoring console 103. The pinch valves 117 and 120 are closed and open, respectively, to drain the fluid into the drainage bag 106.

FIG. 10 shows a block diagram illustrating the entire system and the connection between the global server (i.e., cloud) 115, the local servers 114, and the monitoring consoles 103.

FIG. 11 illustrates the block diagram of the inputs and outputs of the global server software 138.

FIG. 12 illustrates the block diagram of the inputs and outputs of the local server software 132.

FIG. 13 illustrates the block diagram of the inputs and outputs of the monitoring console software 127.

FIG. 14 and FIG. 15 provide process flow diagrams showing the different operational modes available in one embodiment of the invention.

FIG. 16 illustrates a depiction of an alternative embodiment of the invention, which includes: 1) a monitoring console 251, including, preferably, a touch display and related interconnecting electronics; 2) at least one remote monitoring device, selected from the group consisting of, in this embodiment, a server 250 (can be a local or/and global) communicating via a wired or preferably wireless network (Wi-Fi); 3) the drainage cartridge enclosure 252; 4) sterilized single-use drainage parts, including but not limited to catheters 257, the tip 259, stopcocks 256 and 258, drainage cartridge 254, collection bag 255, which are user-installable and replaceable.

FIG. 17 presents a prototype of the invention which includes: the monitoring console 300, the drainage cartridge enclosure 305, the cables 302, and 303 connect the monitoring console 300 to the drainage cartridge enclosure 305, the drainage bag 301, the cartridge 307, the stopcock 308, and the DC motor 304 to control the stopcock.

FIG. 18 shows the back view and inside of the monitoring console 300 which includes the Wi-Fi antenna 310, a 10-inch touch display 312, a connector 311, and a processor unit 314.

FIG. 19 illustrates the back view and inside the drainage cartridge enclosure 305 which includes the connection cables 302 and 303, the connector 319, the array of light-emitting diodes 318, and the camera 317.

### DETAILED DESCRIPTION OF THE PREFERRED EMBODIMENTS OF THE INVENTION

A detailed and enabling written description of the preferred embodiments of this invention is provided herein, including through the various drawings which constitute a part of this specification and include non-limiting exemplary embodiments of the invention, which may be embodied in various alternate or equivalent forms. It is to be understood that specific details disclosed herein are not to be interpreted as limiting, but rather as a basis to employ the present invention to monitor, manage, and control virtually any appropriate drainage system now known which hereafter comes into being.

Note that, throughout, like elements shown in the drawings of various views of the invention are similarly numbered, although not all such elements shown in all such views are required to be present in every embodiment of the invention, nor should this convention be understood to exclude or imply that every view shown in the figures is of the same embodiment of the invention. The foregoing applies, of course, except where, in a given context, it is clear that such elements belong together through the cooperative engagement of such elements or because such elements are specifically referred to in a given embodiment.

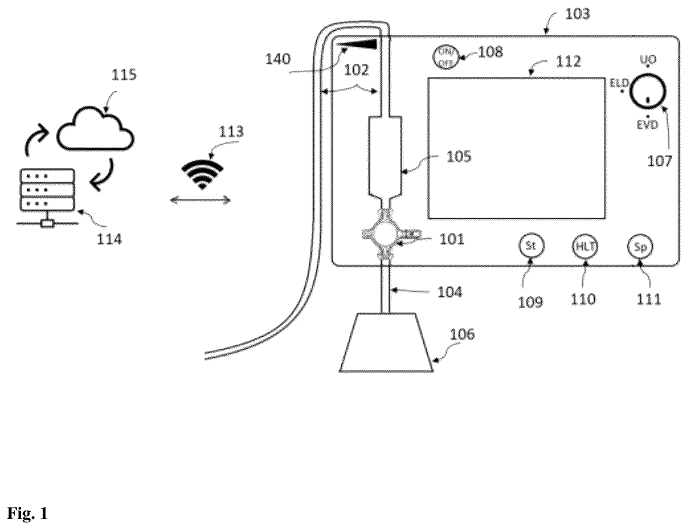

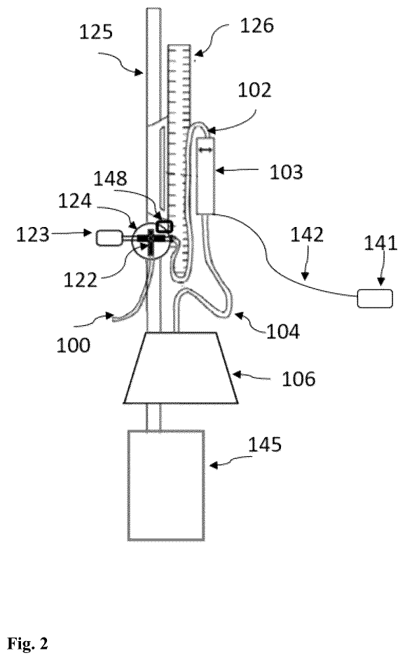

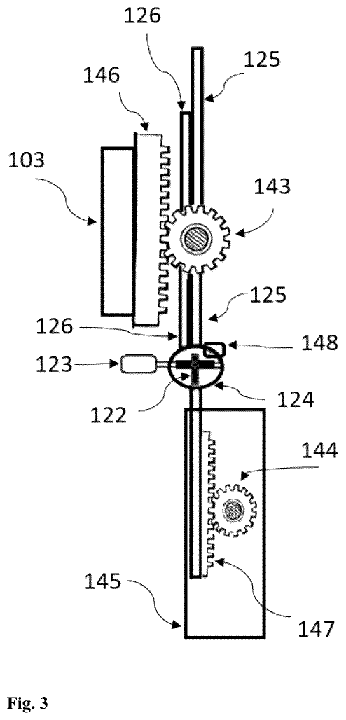

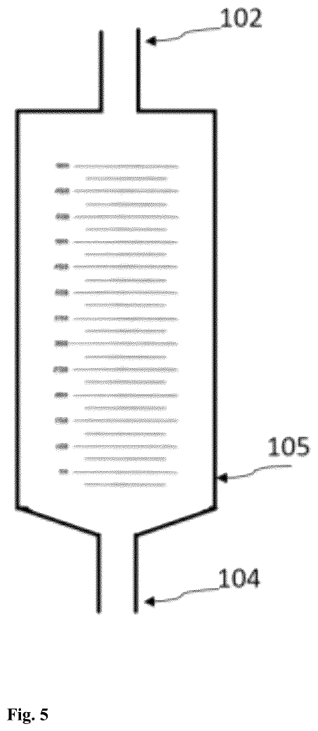

Referring first to FIG. 1, shown in front view, in one embodiment of the present invention, a system is shown for implementation of this invention in management and control of e.g., CSF, urine drainage, or similar applications. The system includes a monitoring console 103, remote devices, including a local server (e.g., an Electronic Health Records (EHR) server) 114, a global or remote server 115 (e.g., a cloud server), or both, and sterilized single-use drainage disposable components typically used in such drainage procedures. The monitoring console 103 is, preferably, a multiple-use, non-sterilized, bedside device utilized to monitor and manage the fluid flow of a patient. Using the knob 107, the monitoring console 103 can be programmed to work for ELD, EVD, UO, or other fluid drainage applications. As the side view of the system shows in FIG. 2, the monitoring console 103 can be clamped and mounted on an IV pole or similar support structure 125 adjacent to a patient. Sterilized single-use drainage components include, but are not limited to, tubing or catheters 100, 102, and 104, stopcocks 101 and 122, a pressure transducer 123, a drainage cartridge 105, (shown in further detail in FIG. 5), and a collection bag 106. The monitoring console 103 relies on gravity to drain fluid out of the body (i.e., from the ventricle, spine, or bladder) via the catheter 100 that goes into the stopcock 122. The stopcocks 101 and 122 could be 2-way or 3-way or 4-way, which, heretofore, are adjusted and controlled manually, but which, according to the present invention, are adjusted and controlled electronically by electric motors, servos, or the like. The fluid drains from the outlet of the stopcock 122 into the drainage cartridge 105 via the catheter 102. The output of the drainage cartridge 105 is connected to the inlet of the stopcock 101. The outlet of the stopcock 101 is connected to the inlet of the collection bag 106 via the catheter 104. The stopcock 101 could have a port for the sampling of the fluid. Referring to FIG. 2, the pressure transducer 123, the stopcock 122, and an electric motor 124 are located and fixed at zero mmHg on a pressure scale panel 126. Based on the application, the “0” reference on the pressure scale panel 126 (i.e., where the pressure transducer 123 is located) should be leveled with the tragus (for EVD applications), the lumbar space (for ELD applications), or iliac crest at the mid-axillary line (for UO applications) for zeroing and calibrating the pressure transducer 123. If needed, a body temperature sensor 141 can be added to monitoring console 103, so the patient's body temperature can be monitored and added to the recorded data for better decision-making. The body temperature sensor 141 is connected to the monitoring console 103 via a wire 142, however, the communication can be wireless as well. The IV pole or similar support structure 125 could be mounted on a base 145. To drain CSF, an indicator 140 may be aligned with the prescribed pressure on the pressure scale panel 126. As shown in FIG. 3, two moving platforms can be utilized to control the ICP and/or flow rate by adjusting the height of the monitoring console 103 or the whole platform (comprising the monitoring console 103, the pressure scale panel 126, the pressure transducer 123, the stopcock 122, the electric motor 124, and gyroscope and altimeter sensors 148) with respect to the patient's tragus or lumbar space. Here two racks and pinions mechanisms are used. The rack and a cylindrical gear referred to as the pinion mechanism serves to transform circular motion into linear motion. However, any other mechanical mechanism can be utilized to function as explained in the following. Gyroscope and altimeter sensors can be attached to the patient's tragus or lumbar space, or iliac crest to detect the orientation and altitude of the patient compared to the pressure transducer 123 (and “0” reference on the pressure scale 126). In this case, other gyroscope and altimeter sensors 148 are fixed at the location of the pressure transducer 123. Based on the data received from the gyroscope and/or altimeter sensors at both locations, the monitoring console 103 determines if the height of itself or the IV pole 125 should be adjusted. This adjustment can be done manually or automatically using the mechanical mechanism (i.e. gears 144, 143 and racks 146, 147) controlled by the monitoring console 103 that moves the whole platform or the drainage cartridge 105. If the patient has moved up or down, the “0” reference on the pressure scale panel 126 (and where the pressure transducer 123 and gyroscope and altimeter sensors 148 are located) should be leveled with the tragus (for EVD applications), the lumbar space (for ELD applications), or iliac crest at the mid-axillary line (for UO applications). In this case, based on the data received from the gyroscope and altimeter sensors at both locations (i.e. the ones attached to the patient and the ones attached to the IV pole 125 or similar support structure) the monitoring console 103 controls the gear 147 to move the IV pole 125 up or down to make the “0” level adjustment. The mechanical mechanism inside the base 145 moves the IV pole 125 or similar structure up or down. In this case, a stationary circular gear 144, rotates clockwise or counterclockwise as needed. The teeth of gear 144 interlocks with those of a moving rack 147. The rack 147 can only move linearly up or down confined to a specific track that moves the IV pole 125 or similar support structure and all attached components to it.

In case the adjustment of the “0” reference is set, if the pressure measured by the pressure transducer 123 deviates from what it is prescribed, the monitoring console 103 can adjust the ICP/abdominal pressure (and consequently adjust the flow rate) by controlling and managing the gear 143. Attached to the IV pole 125 or similar support structure, is a stationary circular gear 143 that rotates clockwise or counterclockwise as needed. The teeth of the gear 143 interlocks with those of a moving rack 146. The rack 146 can only move linearly up or down and is confined to a specific track. The monitoring console 103 is attached to the rack 146. This mechanism just moves the monitoring console 103 up or down. To drain urine for the UO application, the drainage cartridge 105 and the collection bag may be placed at a height below the patient's iliac crest.

The user may follow the health center protocols and the pressure transducer 123 user manual instructions to make reliable ICP or intra-abdominal pressure measurements. The pressure transducer 123 can be inline or offline the drainage path using the stopcock 122. The stopcock 122 can be controlled manually by the user or automatically by the electric motor 124. The pressure transducer 123 measures ICP or intra-abdominal pressure continuously or at desired occasions based on the application and healthcare professional preference. The monitoring console 103 is connected to the electric motor 124 via a cable to control the stopcock 122 as programmed by the user based on the application. For ELD applications, the electric motor 124 can control the stopcock 122 and stop the flow into the drainage cartridge 105 if a specific amount of CSF is desired to be drained per hour. Fluid drains and accumulates into the drainage cartridge 105, where the fluid's flow rate, volume, and color are measured.

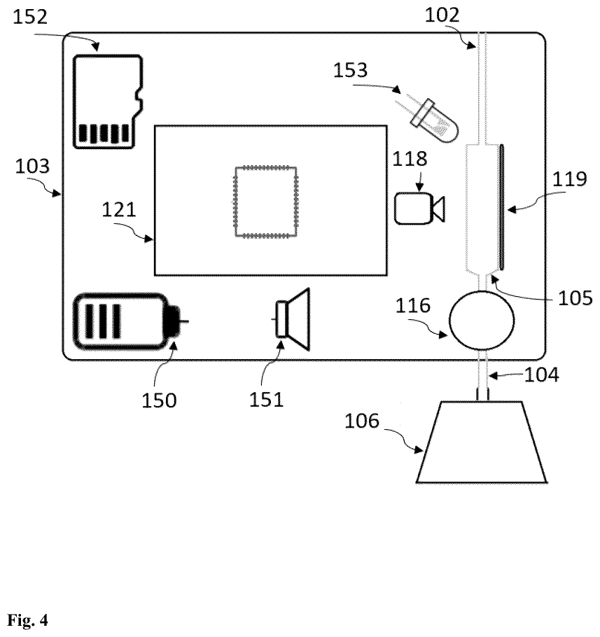

In one preferred embodiment, inside the monitoring console 103 (rear view without the back enclosure) is shown in FIG. 4. In the configuration shown in this figure, stopcock 101 is closed to the fluid flow into the collection bag 106 to allow fluid accumulation into the drainage cartridge 105. To control the flow, an electric motor 116 controls stopcock 101 as programmed by the user. Once the drainage cartridge 105 is full, stopcock 122 stops the fluid flow into the drainage cartridge 105 while the stopcock 101 allows fluid drainage into the collection bag 106. The monitoring console 103 can activate an alarm using speaker 151 to inform health professionals in emergency situations.

Processing unit 121 is a small form factor computer that includes, but is not limited to, a processor, memory, Wi-Fi module, and/or other peripherals. The monitoring console 103, is preferably portable and, preferably, in one embodiment, is operable in a standalone mode to capture real-time data from the camera 118, pressure transducer 123, or other integrated peripherals. Data shown on display 112, in one embodiment, is sent to the local server 114 (i.e., EHR) over, for example, a Wi-Fi network 113, or equivalent means of communication, including, e.g., via a wired local area network. In a preferred embodiment, the local server 114 sends data to a global server 115 via the internet, via wireless, or equivalent communication means, including e.g., via a wired connection, mixed with Wi-Fi servers and combinations thereof, or other communication means which hereafter come into being.

The monitoring console 103 is powered from a power outlet, or via other wired or wireless power delivery means. In a preferred embodiment, the monitoring console 103 is equipped with an internal rechargeable battery 150 in case of an external power supply outage. In the case of Wi-Fi or other data communication means 113 disconnection, all real-time data are stored and backed up on a local memory card 152 to avoid data loss. Once the Wi-Fi 113 connection is reestablished, the monitoring console 103 automatically sends recorded data during the Wi-Fi 113 disconnection to the local server 114.

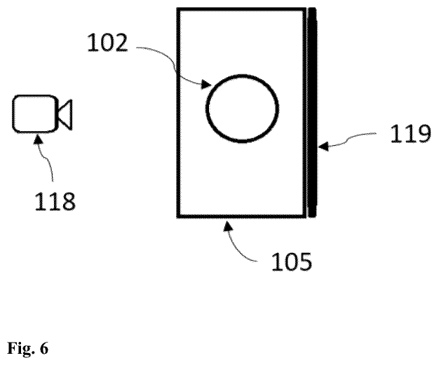

In the view of an embodiment of this invention depicted in FIG. 6, the top view of a drainage cartridge 105, heating element 119, and camera 118, is shown. The shape of the drainage cartridge 105, in this embodiment, is rectangular to increase sensitivity. However, the shape of the drainage cartridge 105 may be cylindrical, triangular, or any other shape. The shape or size of the drainage cartridge 105 can be adjusted or selected to achieve high accuracy and optimal performance. Depending on the application, camera 118 could be a visible and/or thermal camera that determines the fluid flow rate from captured images. A visible camera relies on visible light to create detailed color images optimal for target identification, while a thermal camera detects heat radiation emitted from an object. For applications where the drainage cartridge 105 remains transparent even for long-term usage, camera 118 could be a visible camera to use computer vision and/or machine learning to monitor the fluid flow rate, clarity, and opaqueness. If needed, a light source 153 or an array of the light source can be used to emit enough and uniform light to the drainage cartridge 105 to improve the image quality of camera 118. On the other hand, for applications where drainage cartridge 105 becomes opaque (so fluid is not visible to the camera) over time, camera 118 could be a thermal camera to detect infrared energy (i.e., heat) reflected from fluid accumulated in the drainage cartridge 105. In this case, heating element 119, in physical contact with drainage cartridge 105, warms up fluid to a specific temperature that is safe for drainage cartridge 105 (e.g., 30 to 45 degrees Celsius, depending on the drainage cartridge 105 material). In this case, since the fluid has much higher thermal conductivity than air, camera 118 captures the higher fluid temperature than the air inside the drainage cartridge 105. Camera 118 can detect the liquid level (i.e., flow rate and volume in real-time) from the higher temperature area in the viewing target area. Camera 118 and the light source 153 or an array of the light source may be positioned at any appropriate location or angle in relation to drainage cartridge 105 to obtain long-term monitoring, high accuracy, and optimal performance. If camera 118 functions as a thermal camera, the color of the fluid may not be detected. The processing unit 121 is wired to the display 112 as shown in FIG. 1, as well as to the electric motors 116, gyroscope and altimeter sensors 148, the pressure transducer 123, and the body temperature sensor 141 as shown in FIG. 2, the battery 150, the light source 153, the heating element 119, and the camera 118 as shown in FIG. 4.

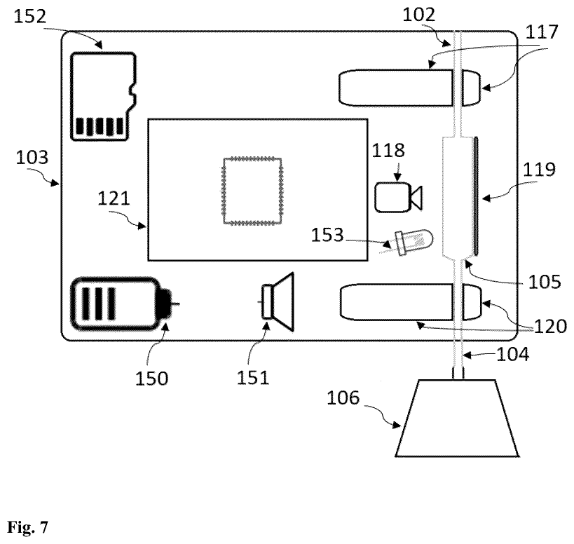

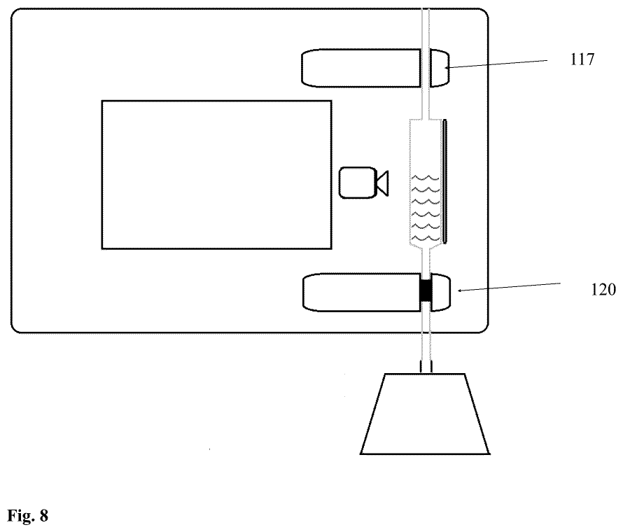

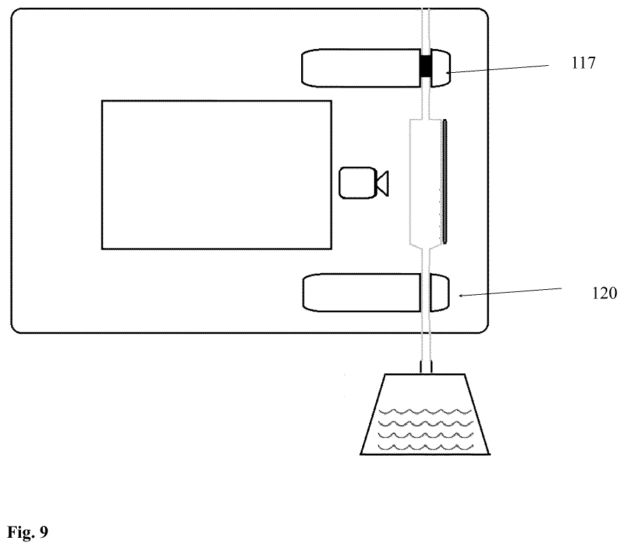

In an alternative embodiment shown in FIG. 7, to control the flow into or from drainage cartridge 105, electric motors 116 and 124 are replaced with pinch valves. In this case, stopcock 122 is controlled manually. The pinch valves regulate, interrupt, or completely stop fluid flow through the catheters 102 and 104. Pinch valves 117 and 120 are normally open and closed, respectively. A normally open valve permits flow in the de-energized state (i.e., without power, gravity induced flow). In contrast, a normally closed valve closes the pinch elements to shut off flow in the de-energized state. When power is applied, the normally open and normally closed valves obstruct and allow fluid flow through the catheter, respectively. For EVD/UO applications, pinch valve 117 is open to allow continuous drainage of CSF/urine into drainage cartridge 105, while pinch valve 120 is closed (see FIG. 8). Once drainage cartridge 105 is full, pinch valve 117 closes, pinch valve 120 opens and drains CSF/urine into collection bag 106 (see FIG. 9), and this cycle repeats. For ELD applications, pinch valve 117 is closed once a specific amount of CSF (predefined conditions programmed in monitoring console 103 by a healthcare professional) is drained into drainage cartridge 105 for a specified period of time. In this case, pinch valve 120 opens and drains CSF into the collection bag 106, and this cycle repeats. For example, the user can program monitoring console 103 to drain 10 mL of CSF per hour from the lumbar spine.

Before any measurement, preferably, camera 118 and pressure transducer 123 are appropriately calibrated. Since the monitoring camera is non-invasive (i.e., it is not in direct contact with fluid), it does not cause occlusion along the drainage path.

One of the most significant potential benefits of this invention is its ability to create essential insights from the tremendous amount of data and enhance predictions of future events at an expert level, and, in some cases, surpass clinicians’ performance. Herein, the invention gathers data from AKI and hydrocephalous patients; however, this invention can be used for other patients or applications.

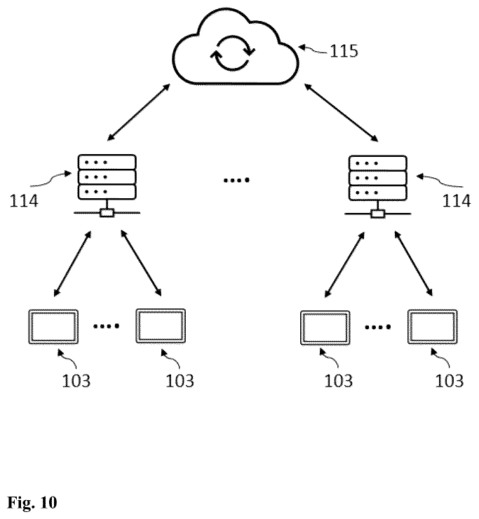

In one embodiment, shown in FIG. 10, one embodiment of an overall system architecture utilizing the present invention includes data exchange between a global server 115, a number of local servers 114, and a number of monitoring consoles 103. An unlimited number of monitoring consoles 103 are connected to local server 114. In addition, there could be any number of local servers 114 connected to global server 115 via the internet or wireless. A local network comprises one local server and all the monitoring consoles that communicate with it. A global network includes global server 115 and all the local servers and monitoring consoles communicate with the global server 115. Global server 115, in one embodiment, is a cloud platform that collects data from all the local servers 114, analyzes data, and provides information to local servers 114.

In a preferred embodiment of the invention, the monitoring, command and control system according to this invention has AI capabilities on both hardware and software levels. The best AI performance is obtained when the local servers 114 and the global server 115 collaborate and exchange data.

The data can be categorized into structured and unstructured data. Structured data are all the measured data, including fluid flow rate, volume, discoloration, intracranial/intra-abdominal pressure, body temperature, or any other relevant parameter measured to ensure tight control over fluid drainage and its effects on the patient.

Unstructured data includes clinical notes, medical reports, articles, and laboratory test reports. The healthcare staff can enter the unstructured data into the software installed on the local server 114. The structured data, the unstructured data, or both, is/are utilized in deep learning to better assist physicians with disease diagnosis and treatment suggestions.

AI software may be developed to use a continuous learning framework implemented on the monitoring console 103, the local server 114, and the global server 115. Every time the monitoring console 103 is used, it learns from its own measured data (i.e., the structured data). Also, each monitoring console learns from other monitoring consoles connected to the same local server 114 and the other monitoring consoles connected to the global server 115 via their corresponding local servers. The AI software continuously logs data (i.e., the structured data and the unstructured data) from many local servers and monitoring consoles, improving real-time collaboration and intelligence functionality.

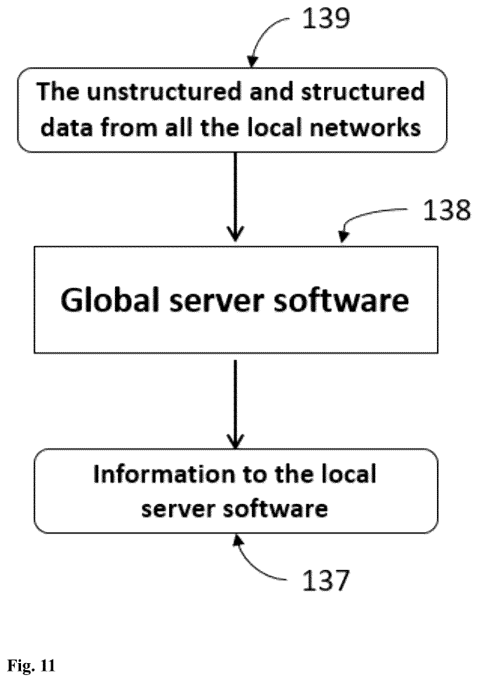

In preferred embodiments, FIGS. 11, 12, and 13 illustrate, in block diagram format, inputs and outputs of three embodiments of AI software that may be developed for this invention comprising the monitoring console and its software 127, a local server and its software 132, and at least one global server with its software 138. The three layers of software are in operative communication with each other.

Referring to FIG. 11, the input of a global server and its software 138 may be the unstructured and the structured data from all the local networks 139. The output of the global server and its software 138 provides information to local servers and their software 137. The information includes unstructured data, structured data, and any decision-making suggestions. The global server software 138 has access to a comprehensive database which it utilizes to continuously improve its performance and decision-making algorithms. The local server software 132 may request a decision-making suggestion from the global server software 138. The global server software 138 may provide a more reliable decision-making suggestion than the suggestion made by the local server software 132 itself.

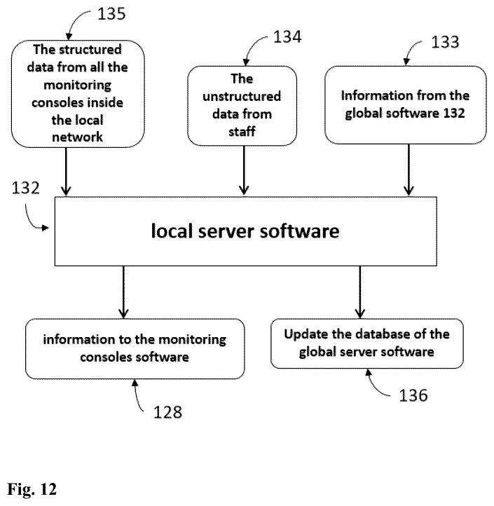

Referring to FIG. 12, the local server software 132 includes three inputs: 1) the structured data from all the monitoring consoles inside the local network 135, 2) the unstructured data from staff 134, and 3) the information from the global server software 133. Users and staff can enter the unstructured data into the local server software 132. The local server software 132 may update the database of the global server software 136 with the unstructured data and/or the structured data of its database. This database consists of the unstructured data and the structured data received from all the monitoring console software 127 inside the local network and/or from the global server software 136. The local server software 132 may send the information (comprising of the unstructured data and/or the structured data and/or the decision-making suggestion) to monitoring console software 128 inside its local network. The monitoring console software 127 may request a decision-making suggestion from the local server software 132. That is, the local server software 132 comprised a more comprehensive database than the monitoring console software 127 and can indicate a more reliable decision-making suggestion. The local server software 132 can operate, analyze data, and make decisions independent of the global server software 138.

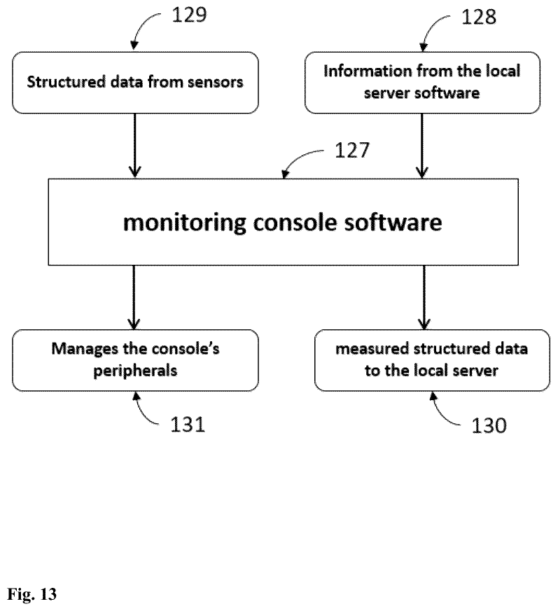

Referring to FIG. 13, the monitoring console software 127 receives two types of inputs. One input is the structured data from sensors 129 (here, the sensors refer to the camera 118, the pressure transducer 123, or the body temperature sensor). The other input is information from the local server 128. The monitoring console software 127 updates the local server database with its structured data and manages the console's peripherals 131 (here peripherals refer to electronics elements consisting of the display 112, the electric motors 116 and 124, the pinch valves 117 and 120, the camera 118, the pressure transducer 123). The monitoring console software 127 is designed to collaborate with the local server software 132. However, the monitoring console software 127 could work as standalone software, analyze data, and make decisions independent of the local server software 132. The monitoring console software 127 provides real-time monitoring of fluid flow rate, fluid volume, ICP or intra-abdominal pressure, and body temperature to healthcare personnel. Over-drainage, under-drainage, occlusion, possible bleeding/infection, Wi-Fi disconnection, and low battery charge generate an alarm on the monitoring console 103, and a corresponding warning will be sent to the local server software 132 via the internet or Wi-Fi 113 for immediate intervention by personnel. Also, the monitoring console software 127 allows the healthcare staff to manually enter the intracranial/intra-abdominal pressure and body temperature.

The physicians and nurses can monitor the real-time data on the display 112 of the monitoring console 103 or using a computer connected to the local server. The local server could be a computer on which the local server software 132 is installed. A mobile application can be developed for personnel's mobile devices if needed for monitoring all data in real-time from anywhere as long as they have access to the internet or Wi-Fi 113.

Different computer vision algorithms can be used to implement the image processing and determine the liquid level inside the drainage cartridge 105. Computer vision algorithms are a subset of AI that enable the monitoring system to understand and interpret visual data from the images and videos taken by the camera. By combining image processing and machine learning techniques, computer vision can be implemented to make decisions based on fluid color changes and flow rates. The camera 118 can be used to capture images or videos of the fluid as it flows into the drainage cartridge 105. Image processing techniques can then be used to extract information from the images, such as the color of the fluid and the rate of flow. For example, color segmentation algorithms can be used to isolate the color of the fluid from the rest of the image, and edge detection algorithms can be used to determine the boundaries of the fluid as it flows and calculate the flow rate by tracking the movement of the fluid in consecutive images. Next, machine learning algorithms can be trained to recognize patterns in the color changes and flow rate of the fluid. Such machine learning algorithms include, but are not limited to, image thresholding, contour detection, template matching, and deep learning. For example, a neural network can be trained to identify specific changes in color or flow rate that are indicative of a particular condition, such as a blockage in the tube or a change in fluid composition. Once the machine learning algorithm has been trained, it can be used to make decisions based on the real-time color changes and flow rate of the fluid.

As an alternative approach, light diffraction can be used to detect fluid level in the drainage cartridge 105. When light passes through the transparent drainage cartridge 105, it can be refracted or bent, which can cause a diffraction pattern to form. By placing the light source 153 or array of light sources at a specific angle and position related to the drainage cartridge 105 and positioning the camera 118 to capture the diffraction pattern, the changes in the medium's refractive index can be detected, which can indicate the presence or absence of a fluid or a change in the fluid level. If the drainage cartridge 105 is empty, the diffraction pattern will be uniform and consistent across the entire length of the drainage cartridge 105. As the fluid level rises inside the drainage cartridge 105, the diffraction pattern will be disrupted, indicating the presence of a fluid and its level.

This invention can be used in a variety of applications, including, but not limited to, in the pharmaceutical industry to monitor the quality of medications or in the food industry to monitor the flow rate of liquids or monitoring the flow rate of chemicals in industrial processes. By leveraging AI technology, the invention has the ability to learn from large patient populations. This means that the more data that is inputted into the monitoring console 103 or the local network, the more accurate its predictions become. This can be particularly useful in scenarios where a patient's medical history is complex, or where there are multiple risk factors to consider. The invention can reduce diagnostic and therapeutic errors and better assist healthcare professionals in enhancing health risk alerts and health outcome predictions.

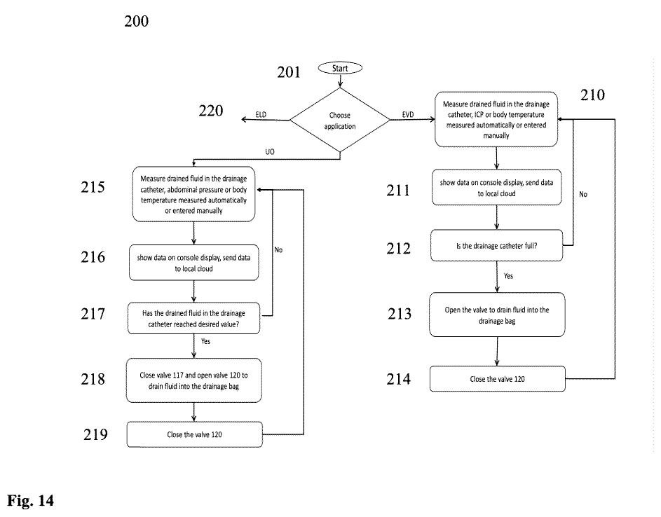

The operation of this device, system, and method may be further understood by reference to FIG. 14, which provides a process flow diagram 200 of the various system operational modes. Starting at 201, in this exemplary embodiment, one of three operational modes is selected, 210 for EVD, 220 for ELD, and 215 for UO monitoring and management. Thus, in this embodiment of the invention, in the event EVD monitoring is the chosen application, at 210, the drained fluid is measured in the drainage catheter or cartridge 105 while ICP or body temperature or both is measured automatically or entered manually. These measurements are displayed on the local console, sent to a cloud server, or both 211. At 212, the system interrogates whether the drainage catheter is full or not. If it is not, process steps 210, 211, and 212 repeat until, at 212, the drainage catheter is full. At that stage, 213, valve 120 is opened and the drainage fluid is drained into the drainage bag. At that stage, 214, valve 120 is closed, and the cycle continues by repeating steps 210-214, until the EVD is terminated.

Similarly, if UO monitoring and management is the chosen application at 201, then the subroutine 215-219 is initiated, rather. Thus, at 215, the amount of drained fluid in the drainage catheter 105 is measured; the abdominal pressure, body temperature, or both are measured automatically or entered manually. These measurements are displayed on the local console, sent to a cloud server, or both 216. At 217, the system interrogates whether the amount of drained fluid in the drainage catheter has reached the desired, that is, pre-defined, amount of drainage. If not, process steps 215, 216, and 217 repeat until, at 217, the determination is made that the desired amount of drainage has been achieved. At that stage, 218, valve 117 is closed, and valve 120 is opened to permit the drained fluid to drain into the drainage bag 106. At that stage, 219, valve 120 is closed, and the cycle continues by repeating steps 215-219, until the urinary fluid drainage is terminated.

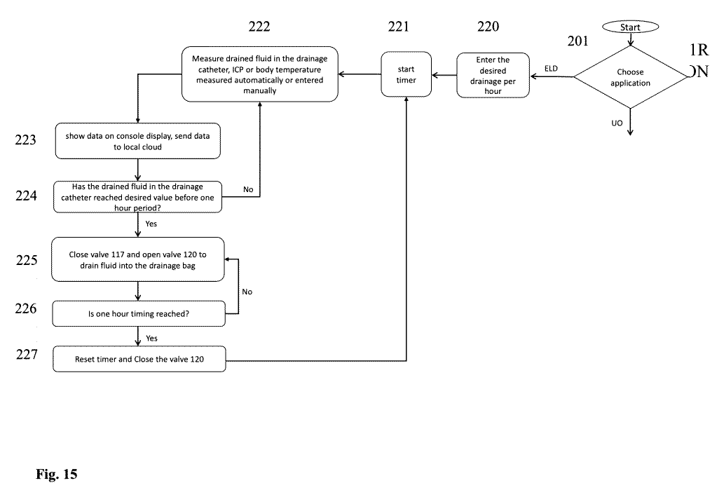

As shown in FIG. 15, where the chosen application is ELD 220 at system start 201, then at 220, the desired rate of drainage per hour is defined. At 221 a timer is initiated, and at 222, the system interrogates the drainage catheter for the total volume drained. In addition, ICP, body temperature, or both are measured automatically or entered manually. These measurements are displayed on the local console, sent to a cloud server, or both 223. At 224, the system interrogates whether the volume in the drainage catheter has reached the desired volume within the defined time period. If it is not, process steps 222, 223 and 224 repeat until, at 224, the desired drainage is met. At that stage, 225, valve 117 is closed, valve 120 is opened and the drainage fluid is drained into the drainage bag 106. At that stage, 226, the system interrogates the timer to confirm the desired time as been reached. If not, the cycle continues by repeating steps 225-226, until the desired elapsed time is reached. At that stage, 227, the timer is reset, valve 120 is closed, and the ELD subroutine is terminated or re-initiated at 221.

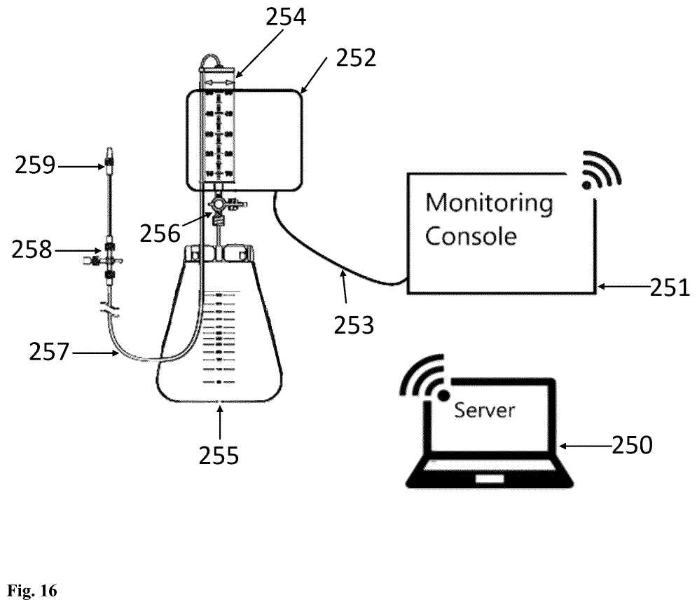

FIG. 16 illustrates a depiction of an alternative embodiment of the invention. In this embodiment, the drainage cartridge is separated from the monitoring console compared to FIG. 1. The monitoring console 251, including, preferably, a touch display and additional electronics wired to the drainage cartridge enclosure 252. At least one remote monitoring device, selected from the group consisting of, in this embodiment, a local or/and global server 250 communicating via a wired or preferably wireless network with the monitoring console 251. The sterilized single-use drainage parts, including but not limited to catheter 257, the stopcocks 256 and 258, the drainage cartridge 254, the collection bag 255, which are user-installable and replaceable. In this embodiment, the IV pole is not shown, however, the monitoring console 251, the drainage cartridge enclosure 252, the drainage cartridge 254, and the drainage bag 255 could be attached to the IV pole. While the monitoring console 251 is fixed at a location, the drainage cartridge enclosure 252 and the drainage cartridge 254 can move vertically on the IV pole to set the pressure at the desired drainage rate. The tip 259 connects to the patient's ventricular or lumbar catheter.

Separating the drainage cartridge from the monitoring console offers the benefit of allowing the enclosure of the drainage cartridge to be readily adapted to accommodate a variety of EVD/ELD/UO devices that are commercially available. Consequently, this design enhances the versatility of the invention by ensuring compatibility with existing single-use EVD/ELD/UO devices without the need to modify or change those devices.

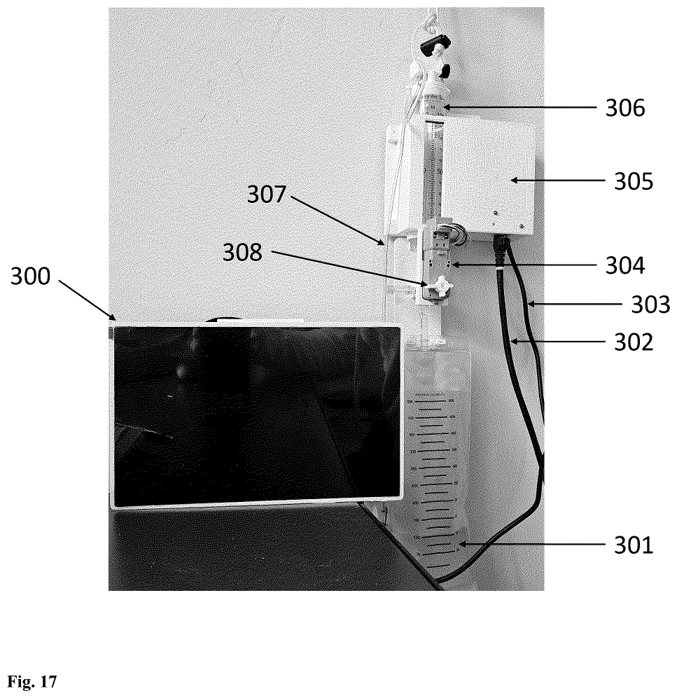

A prototype of the invention is shown in FIG. 17. The monitoring console 300 is a 10-inch touch display wired to the drainage cartridge enclosure 305 using the cables 302 and 303. The drainage cartridge 306 is inserted from the back of the drainage cartridge enclosure 305. The open slot at the front of the drainage cartridge enclosure 305 makes the drainage cartridge 306 visible to health professionals. There is a stopcock between the collection bag 301 and the drainage cartridge 306 that is not visible in this figure (it is behind the DC motor 304). The stopcock 308 is attached to the single-use stopcock located between the collection bag 301 and the drainage cartridge 306 as the DC motor is double-shaft. In this case, the stopcock is controlled automatically using the DC motor 304 or manually by the user. The DC motor 304 is equipped with an encoder sensor to send the exact rotational position of the stopcock to the monitoring console to provide extra safety. The local and/or global servers are not shown in this figure.

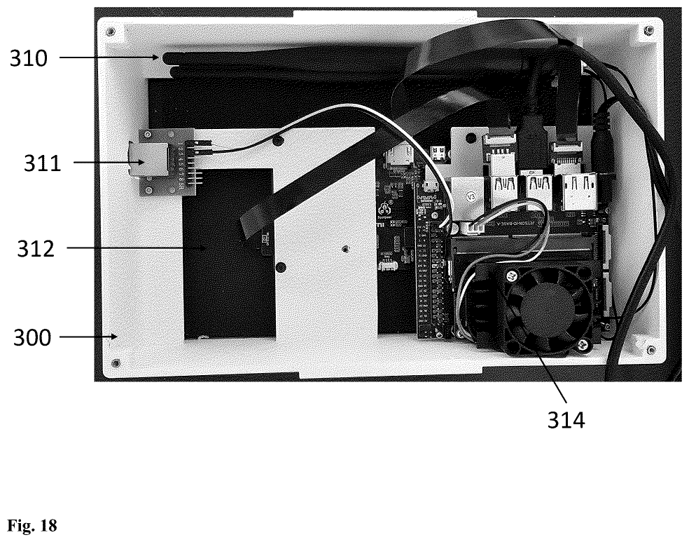

FIG. 18 shows the back view and inside the monitoring console 300. The processor unit 314 includes a processor, memory, Wi-Fi module, different types of connectors, among others. The antenna 310 sends and receives data to/from the local and/or global servers.

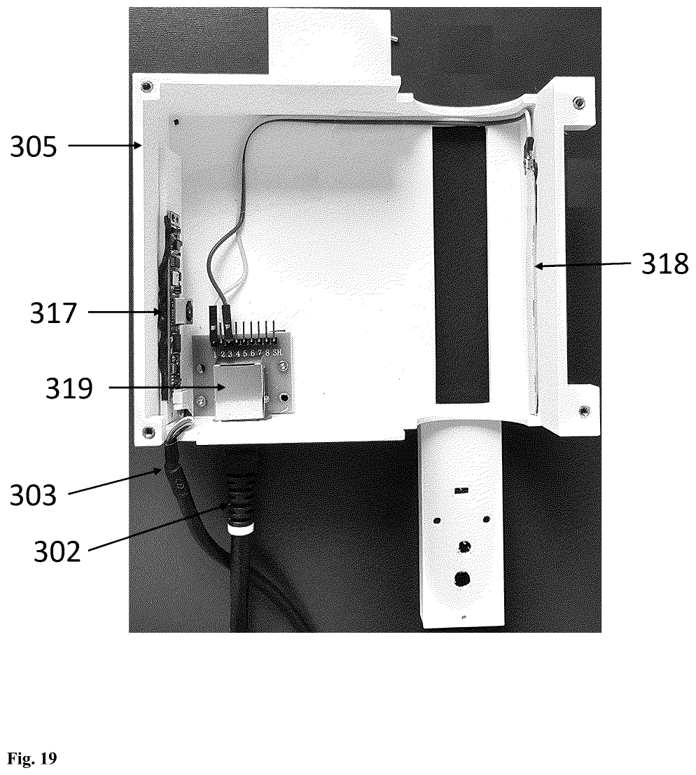

FIG. 19 illustrates the back view and inside the drainage cartridge enclosure 305. The cables 302 and 303 connect the camera 317, the array of light-emitting diodes 318 (to provide uniform lighting), and the DC motor 304 to the monitoring console. It is not shown here but the DC motor connects to the connector 319.

Having described this invention, including how to make and use the system, method and device of this invention, those skilled in the art will appreciate that this invention, including equivalents thereof, provides distinct advantages over known methods, systems, and devices. Typically, such known systems depend on double pressure measurement and differential to control said fluid drainage equipment. Such methods and equipment, form no part of the present invention which may be used in addition to or which may completely supplant such systems, methods and devices.

The present invention provides a fluid drainage monitoring, management, and control system which includes:
fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

The equipment is adapted for drainage of fluid in at least one of ELD, EVD, and UO, and preferably includes at least one sensor of fluid clarity, color, flow rate, viscosity, volume, composition, temperature, or combinations thereof, which, when pre-set clarity, color, temperature, or composition parameters are exceeded, the system adjusts fluid flow accordingly to bring said parameter back within acceptable limits.

The sensor senses fluid present in a drainage component, such as a fluid containing cartridge of defined volume, to thereby ensure relevant desired drainage parameters remain within desired limits. Preferably, the cartridge is translucent to permit a camera to monitor the volume and other parameters of fluid entering and leaving the cartridge. The volume of fluid in the cartridge, including, optionally, the color or composition thereof, is intermediately interrogated by the system to maintain drainage parameters within desired limits.

Referring to the control equipment, this includes at least one of each of the following pieces of equipment operably connected to each other:
a processing unit; and
a sensor of said physiological parameter.

Preferably, the system is adapted for contact with a living human or non-human animal to provide effectively real-time measurement data reflective of said at least one fluid drainage parameter. Preferably, the effectively real-time measurement data is provided to a processing unit in, effectively, real-time. The processor processes the data by at least one of the following: algorithmic analysis, storage, visual display, and communication to at least one other processing unit. Algorithmic analysis preferably includes production of effectively real-time information on the current physiological state of said living human or non-human animal. This is achieved, in a preferred embodiment, by acquiring data by more than one sensor, which is reflective of multiple different physiological parameters, processed simultaneously or sequentially by more than one processing unit, included in a large database of like data generated from many monitoring consoles in a network, and which is utilized as a training set for at least one processor to predict or prevent at least one medical condition selected from the group consisting of: vascular occlusion, over-drainage, under-drainage, infection, presence of blood in said fluid when blood is not the measured fluid.

A fluid drainage monitoring, management, and control device according to the invention includes but is not limited to:
fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

A method for fluid drainage monitoring, management, and control according to the invention includes:
Providing fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
Providing control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

What is claimed is:

1. A fluid drainage monitoring, management, and control system comprising:
a. fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
b. control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

2. The system according to claim 1 wherein said control equipment further comprises at least one sensor of fluid clarity, color, composition, temperature, or combinations thereof, which, when pre-set clarity, color, temperature, or composition parameters are exceeded, the system adjusts fluid flow accordingly to bring said parameter back within acceptable limits.

2. The system according to claim 2 wherein said sensor senses fluid present in a drainage component to thereby ensure relevant desired drainage parameters remain within desired limits.

3. The system according to claim 3 wherein said drainage component comprises a fluid containing cartridge of defined volume.

4. The system according to claim 4 wherein the cartridge is translucent.

5. The system according to claim 5 wherein the volume of fluid in said cartridge, including, optionally, the color or composition thereof, is intermediately interrogated by the system to maintain drainage parameters within desired limits.

6. The fluid drainage control system according to claim 1, wherein said control equipment comprises at least one of each of the following pieces of equipment operably connected to each other:
iii. a processing unit; and
i. a sensor of said physiological parameter.

8. The fluid drainage control system according to claim 1, wherein said fluid drainage equipment is adapted for drainage of fluid in at least one of ELD, EVD, and UO.

7. The fluid drainage control system according to claim 2 wherein said sensor is adapted to measure at least one fluid parameter selected from the group consisting of the: color, flow rate, viscosity, volume, and temperature of a physiological fluid.

8. The fluid drainage control system according to claim 9 wherein said sensor is adapted for contact with a living human or non-human animal to provide effectively real-time measurement data reflective of said at least one fluid drainage parameter.

9. The fluid drainage control system according to claim 10 wherein said effectively real-time measurement data is provided to said processing unit in, effectively, real-time.

10. The fluid drainage control system according to claim 11 wherein said processing by said processing unit is selected from, the group consisting of at least one of the following: algorithmic analysis, storage, visual display, and communication to at least one other processing unit.

11. The fluid drainage control system according to claim 12 wherein said algorithmic analysis provides effectively real-time information on the current physiological state of said living human or non-human animal.

12. The fluid drainage control system according to claim 13 whereby said data: (i) is provided by more than one sensor, (ii) is reflective of multiple different physiological parameters, (iii) is processed simultaneously or sequentially by more than one processing unit, (iv) is included in a large database of like data generated from many monitoring consoles in a network, (v) is utilized as a training set for at least one processor to predict or prevent at least one medical condition selected from the group consisting of: vascular occlusion, over-drainage, under-drainage, infection, presence of blood in said fluid when blood is not the measured fluid.

13. A fluid drainage monitoring, management, and control device comprising:
c. fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
c. control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

16. A method for fluid drainage monitoring, management, and control comprising:
e. Providing fluid drainage equipment adapted for drainage of fluid in at least one medical condition in which fluid drainage is known to be prophylactically, diagnostically, or therapeutically beneficial, within pre-defined prophylactic, diagnostic, or therapeutic drainage limits, to a living human or non-human animal suffering from or being susceptible to said medical condition; and
d. Providing control equipment for controlling said fluid drainage equipment in, effectively, real-time, to achieve precise and accurate control over at least one physiologically affected parameter by said fluid drainage equipment to maintain said affected parameter within said pre-defined prophylactic, diagnostic, or therapeutic drainage limits, wherein said control equipment does not depend on double pressure measurement and differential to control said fluid drainage equipment.

17. The system according to claim 1 wherein said drainage cartridge is housed separately from said monitoring console such that said system may be readily adapted to accommodate a variety of commercially available single-use and manual EVD/ELD/UO devices.
